(19) United States
(12) Patent Application Publication
(10) Pub. No.: US 2017/0246021 A1
(43) Pub. Date: Aug. 31, 2017

(54) Traction system and related methods

(71) Applicant: Jason Orion Jaeger, Las Vegas, NV (US)

(72) Inventor: Jason Orion Jaeger, Las Vegas, NV (US)

(21) Appl. No.: 15/530,629

(22) Filed: Feb. 10, 2017

Related U.S. Application Data
(60) Provisional application No. 62/389,211, filed on Feb. 18, 2016.

(51) Int. Cl. A61F 5/042; A61H 1/02

(52) U.S. Cl. CPC A61F 5/042; A61H 1/0296; A61H 1/0292; A61H 2205/04; A61H 2203/0425

(57) ABSTRACT

The present invention is generally directed to corrective spinal traction systems and methods. It is further directed to use of such spinal traction systems to treat various medical problems associated with abnormal spinal curvature. In one aspect, the present invention is directed to a spine corrective traction system. The system comprises an overhead system allowing a patient's spine to be pulled in every and any vector the human spine moves in three dimensional space, and in two to three planes simultaneously. It further includes a seat assembly that allows for a patient to be seated upright, supine and every angle and vector between 90 degrees upright and 0 degrees horizontal.

### FIELD OF THE INVENTION

The present invention is generally directed to corrective spinal traction systems and methods. It is further directed to use of such spinal traction systems to treat various medical problems associated with abnormal spinal curvature.

### BACKGROUND OF THE INVENTION

There have been reports of spinal traction systems. U.S. Pat. No. 8,882,802, for instance, is allegedly directed to the following: “A chiropractic machine is disclosed which uses separate supporting members for the various body parts of a patient. These separate supporting members are mounted on a flexible structure that allows them to be rotated and elongated in relation to each other. This allows greater flexibility and range of motion, thus facilitating chiropractic treatment.” Abstract.

U.S. Pat. No. 8,152,747 is allegedly directed to the following: “The current invention is a spinal and upper cervical impulse treatment device and controller, which delivers multiple impulses of variable frequency and variable force in a linear direction, as well as rotational forces, for patient treatment. Known chiropractic impulse devices are all hand held devices. In contrast, the spinal and upper cervical impulse treatment device is mounted on a fixed stand and armature, allowing reliable positioning and directional alignment in three dimensions. Fixed mounting also facilitates ease of use. A safety coupling is incorporated to avoid patient injury due to excessive force on the treatment site in the fixed mounting scenario Smooth sinusoidal waveforms are a preferred waveform for impulse delivery and sine waves are generated digitally in the apparatus. Data validation is used to ensure correct directional alignment prior to device activation Patient safety and consistency in treatment protocols are considered in the spinal and upper cervical impulse treatment design.” Abstract.

US Pat. Pub. No. 2014/0188169 is allegedly directed to the following: “The invention provides a cordless chiropractic adjustment device including a thrust element (80) capable of impacting a body contact member (10), a resilient spring (314) arranged to bias the thrust element towards the body contact member, and a motor (120). The motor is arranged to move the thrust element between a variable primed configuration in which the thrust element is held out of contact with the body contact member, and a fired configuration in which the thrust element is propelled by the resilient spring into contact with the body contact member through a range of different impact forces. The motor is provided with direct current by one or more batteries (150), which may be rechargeable.” Abstract.

Despite reports of spinal traction systems, there is still a need for novel, corrective spinal traction systems.

### BRIEF DESCRIPTION OF THE FIGURES

FIG. 1 shows a first traction system (100) according to the present invention.

FIG. 2 shows a first traction system (100) according to the present invention from different views.

FIG. 3 shows a second traction system (300) according to the present invention.

FIG. 4 shows a set of components of a second traction system according to the present invention (top image) and the disconnected base of that system (bottom image).

FIG. 5 shows a vertical leg (500) of a second traction system according to the present invention (left image) and a top tube (502) of the system (right image).

FIG. 6 shows a square tube corner (602) of a second traction system according to the present invention (top image) and a first pulley type (bottom image).

FIG. 7 shows a second pulley type of a second traction system according to the present invention (top image) and a third pulley type (604, bottom image).

FIG. 8 shows a seat back support of a second traction system according to the present invention (800, top image), a large spreader bar (802, middle image) and a small spreader bar (804, bottom image).

FIG. 9 shows a seat back (900) of a second traction system according to the present invention.

FIG. 10 shows different views of a seat assembly (1000) of a second traction system according to the present invention.

FIG. 11 shows two different adjustable positions of a seat assembly (1100) of a second traction system according to the present invention.

FIG. 12 shows a pivot assembly (1200, top image) and a seat back platform (1202, bottom image) of a seat assembly of a second traction system according to the present invention.

FIG. 13 shows a sliding assembly (1300, top image) and a locking assembly (1302, bottom image) of a seat assembly of a second traction system according to the present invention.

FIG. 14 shows a seat platform (1400, top image) and a leg platform (1402, bottom image) of a seat assembly of a second traction system according to the present invention.

### SUMMARY OF THE INVENTION

In one aspect, the present invention is directed to a spine corrective traction system. The system comprises an overhead system allowing a patient's spine to be pulled in every and any vector the human spine moves in three dimensional space. It further includes a seat assembly that allows for a patient to be seated upright, supine and every angle and vector between 90 degrees upright and 0 degrees horizontal.

In another aspect, the present invention provides a method of correcting cervical or lumbar lordosis in a patient such that the normal range for lordosis is achieved. The method comprises the steps of: wrapping the patient in a harness, where the patient is standing, supine or seated at an angle less than 90 degrees; pulling the harness in more than one vector at the same time for a period greater than 10 minutes, not to exceed 20 minutes; releasing the harness; repeating steps “a”, “b” and “c” at least three per week for 12 weeks; thereby achieving a normal range of cervical or lumbar lordosis in the patient.

In another aspect, the present invention provides a method of treating thoracic hyperkyphosis which has been associated with pulmonary and cardiac hypertension, as well as early morbidity in a patient. The method comprises: wrapping the patient in a harness, where the patient is standing, supine or seated at an angle less than 90 degrees; pulling the harness in more than one vector at the same time for a period greater than 10 minutes, not to exceed 20 minutes; releasing the harness; repeating steps “a”, “b” and “c” at least once per week for one month; thereby reducing the patient's thoracic hyperkyphosis which has been associated with pulmonary and cardiac hypertension, and early morbidity, by at least 10 percent.

In another aspect, the present invention provides a method of increasing a patient's lung capacity. The method comprises: wrapping the patient in a harness, where the patient is standing, supine or seated at an angle less than 90 degrees; pulling the harness in more than one vector at the same time for a period less than 10 minutes; releasing the harness; repeating steps “a”, “b” and “c” at least once per week for one month; thereby increasing the patient's lung capacity by at least 10 percent.

In another aspect, the present invention provides a method of decreasing the cost of treating a patient having abnormal cervical spine lordosis or lumbar lordosis relative to surgical intervention. The method comprises: wrapping the patient in a harness, where the patient is standing, supine or seated at an angle less than 90 degrees; pulling the harness in more than one vector at the same time for a period less than 10 minutes; releasing the harness; repeating steps “a”, “b” and “c” at least once per week for one month; thereby restoring cervical spine lordosis or lumbar lordosis to a normal range, and whereby the cost of performing the method is less than ¼^{th} the cost of surgical intervention.

### DETAILED DESCRIPTION OF THE INVENTION

The present invention is generally directed to corrective spinal traction systems. The systems will be described below in relation to figures that depict them.

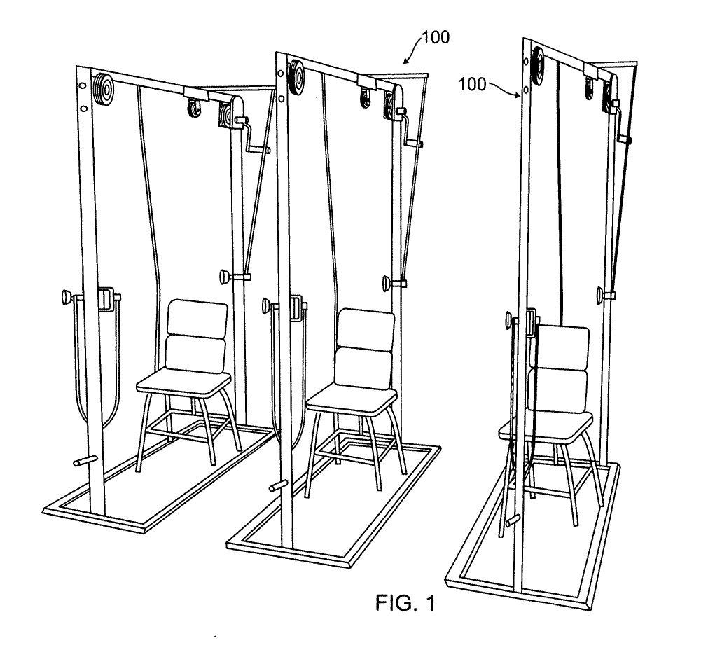

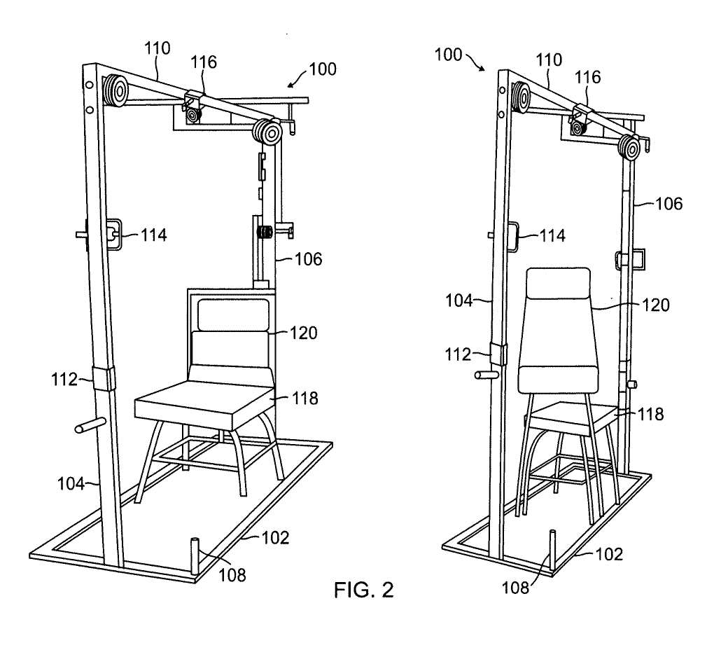

FIG. 1 shows a first traction system (100) according to the present invention. FIG. 2 shows a first traction system (100) according to the present invention from different views. The traction system includes a base (102) to which front (104) and back (106) vertical upright bars are attached, as well as a weight storage pole (108). A top horizontal bar (110) connects the front (104) and back (106) vertical upright bars. Lower pulleys (112) and upper pulleys (114) are attached to the front (104) and back (106) vertical upright bars. An adjustable, removable pulley (116) is connected to the top horizontal bar (110). A chair (118) with an adjustable back (120) is positioned within the system base (102). Cables are attached to the various pulleys such that the first traction system (100) is operational.

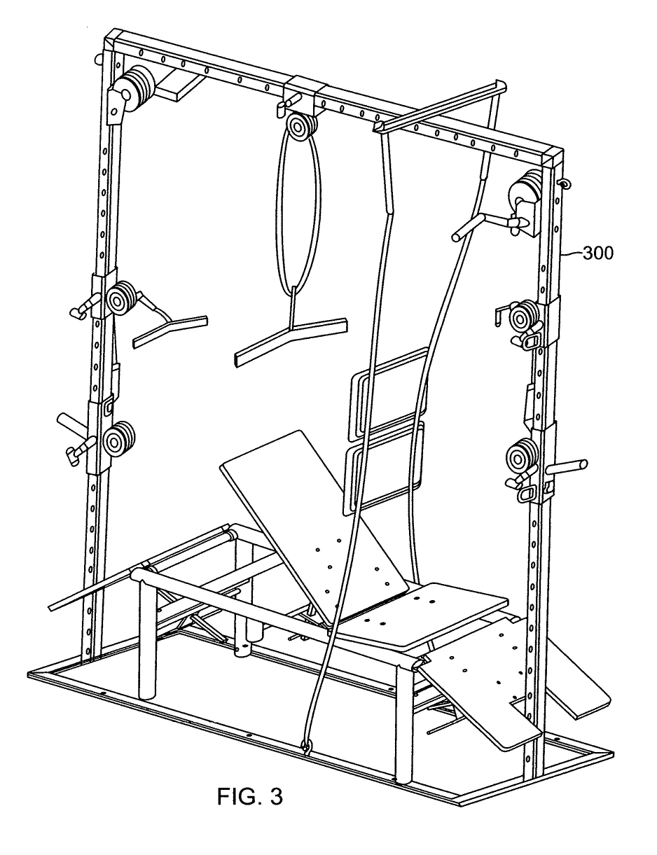

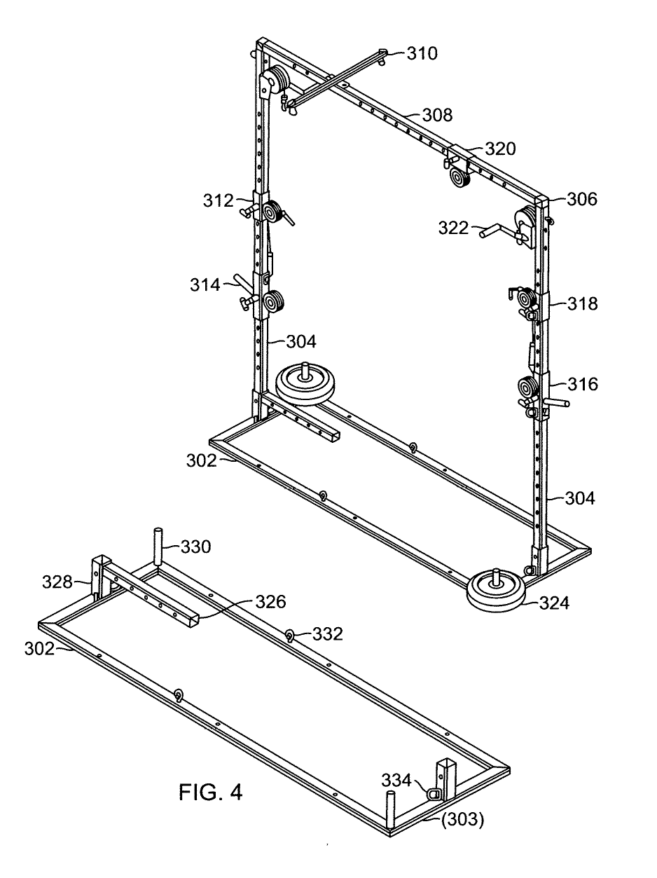

FIG. 3 shows a second traction system (300) according to the present invention. As shown in the top image of FIG. 4, the system includes a framework. As shown, the second traction system comprises the following elements: base frame assembly (302); vertical legs (304); square tube corner (326); top tube (308); seat back support assembly (310); left hand small slide assembly (312); left hand weight support assembly (314); right hand weight support assembly (316); right hand small slide assembly (318); axial slider assembly (320); winch assembly (322). Removable weights (324) are also shown.

FIG. 4 also shows the disconnected base of the second traction system (bottom image). Pieces (302) and (303) are rectangular tubes. Frame support (328) connects bottom horizontal support (326) to rectangular tube (303). Weight support (330) is attached to the corner formed by the attachment of rectangular pieces (302) and (303). Ring mount (332) is connected to rectangular tube (303), and D ring (334) is attached to rectangular piece (303).

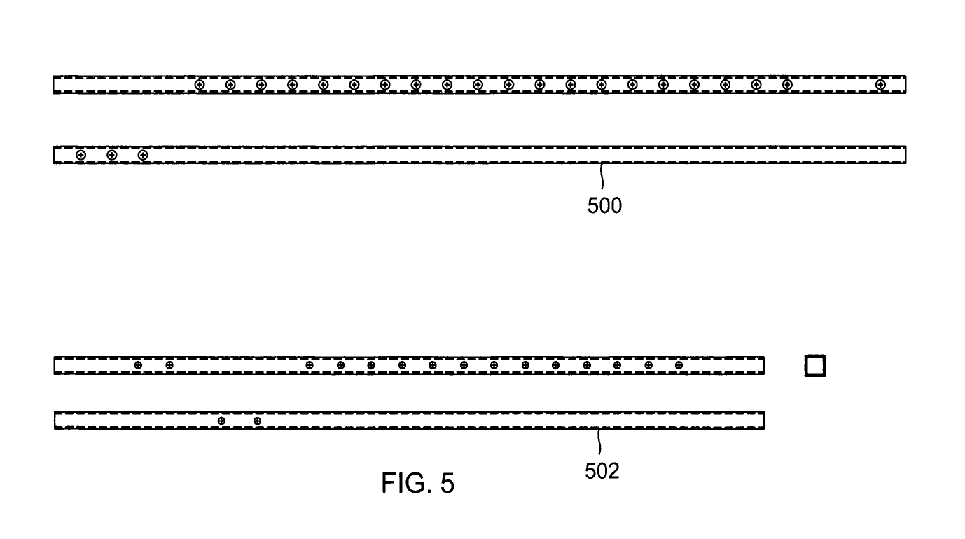

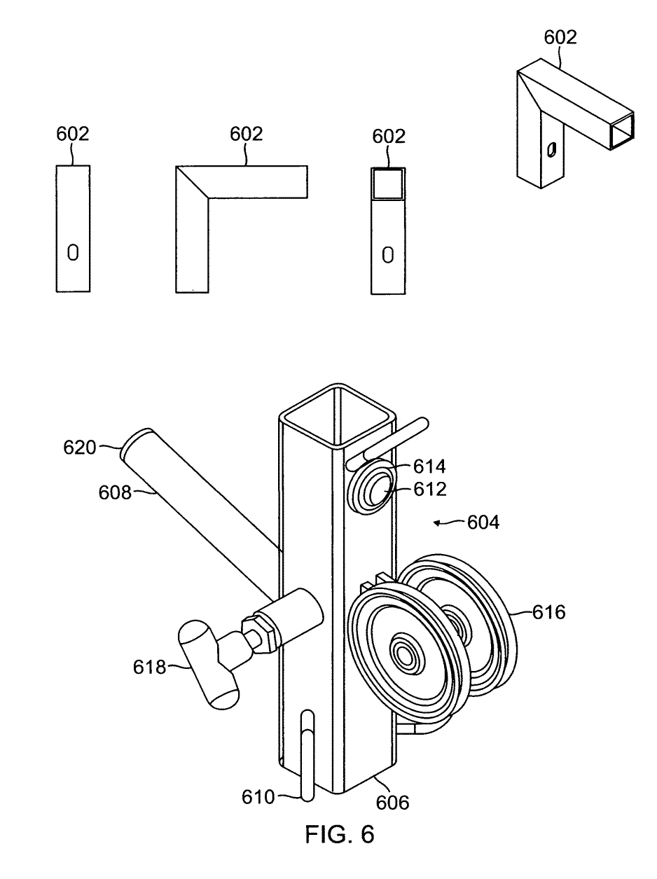

FIG. 5 shows a vertical leg (500) of a second traction system according to the present invention (left image) and a top tube of the system (502, right image). FIG. 6 shows a square tube corner (602) of a second traction system according to the present invention (top image) and a first pulley type (604, bottom image). The first type pulley includes weight slide right hand (606) to which is connected weight support angle (608), D ring (610), Bearing (612), Bearing washer (614), pulley (616), and lock handle (618). Plug (620) is connected to the end of weight support angle (608).

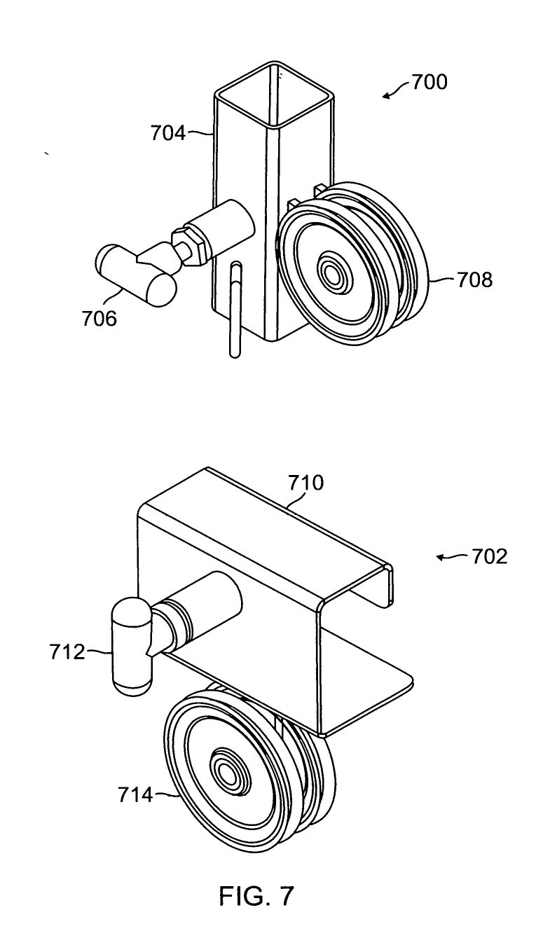

FIG. 7 shows a second pulley type of a second traction system according to the present invention (700, top image) and a third pulley type (702, bottom image). The second pulley type includes small slide (704), to which is attached lock handle (706) and pulley (708). The third pulley type comprises axial slide (710), non-locking handle (712) and pulley (714).

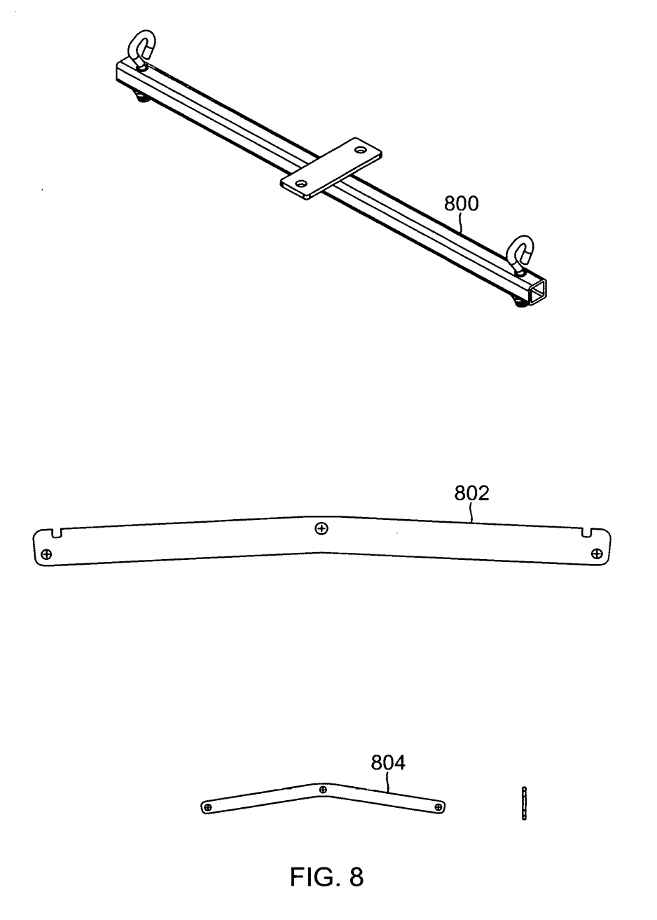

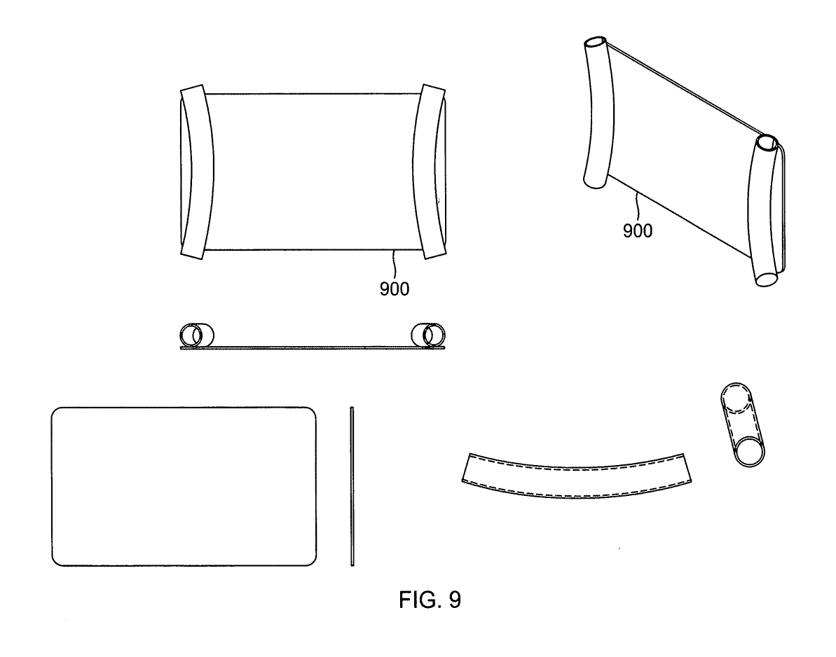

FIG. 8 shows a seat back support of a second traction system according to the present invention (800, top image), a large spreader bar (802, middle image) and a small spreader bar (804, bottom image). FIG. 9 shows a seat back (900) of a second traction system according to the present invention.

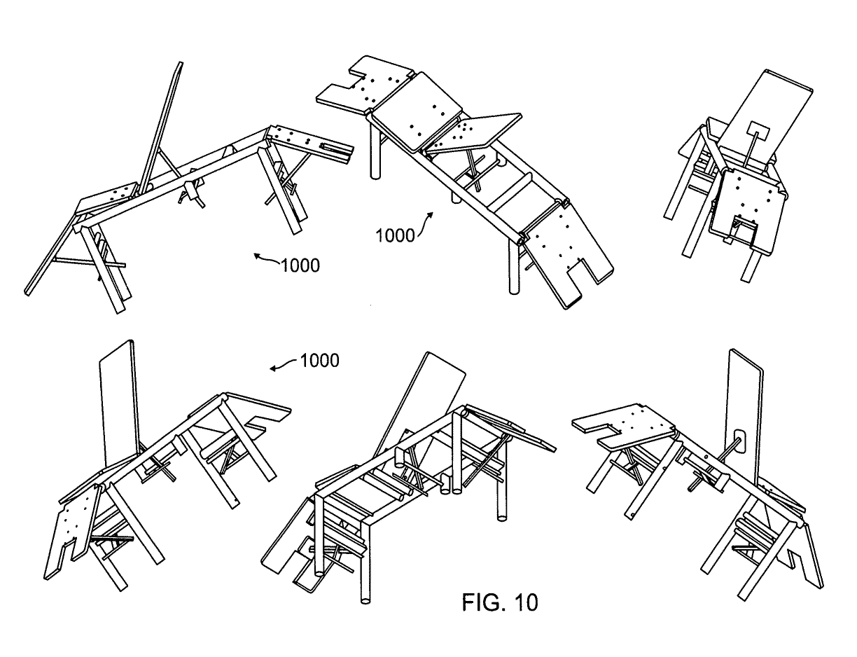

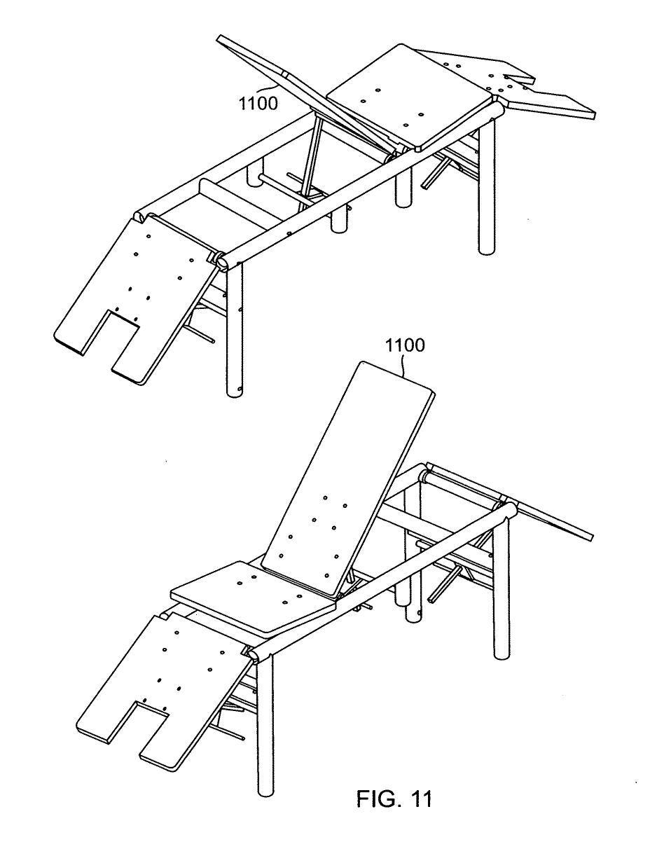

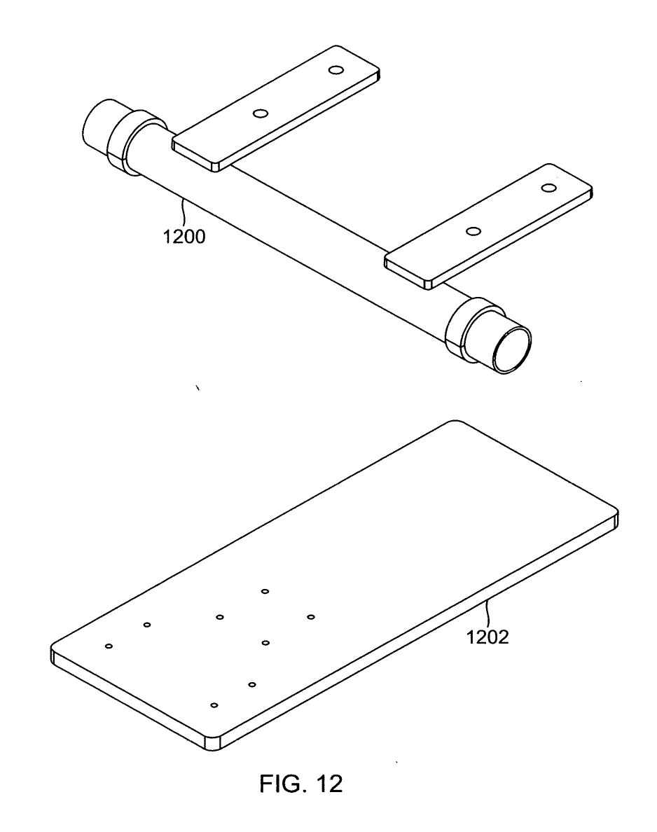

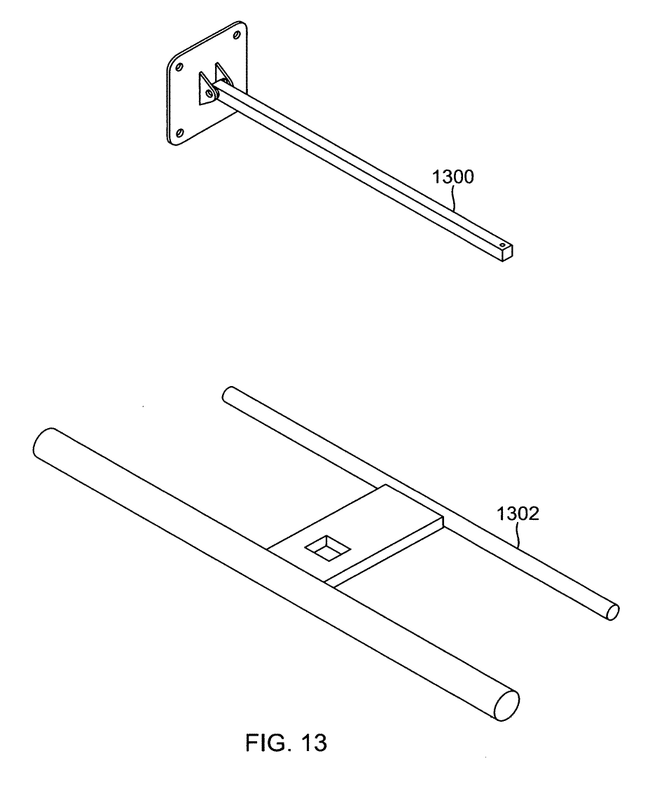

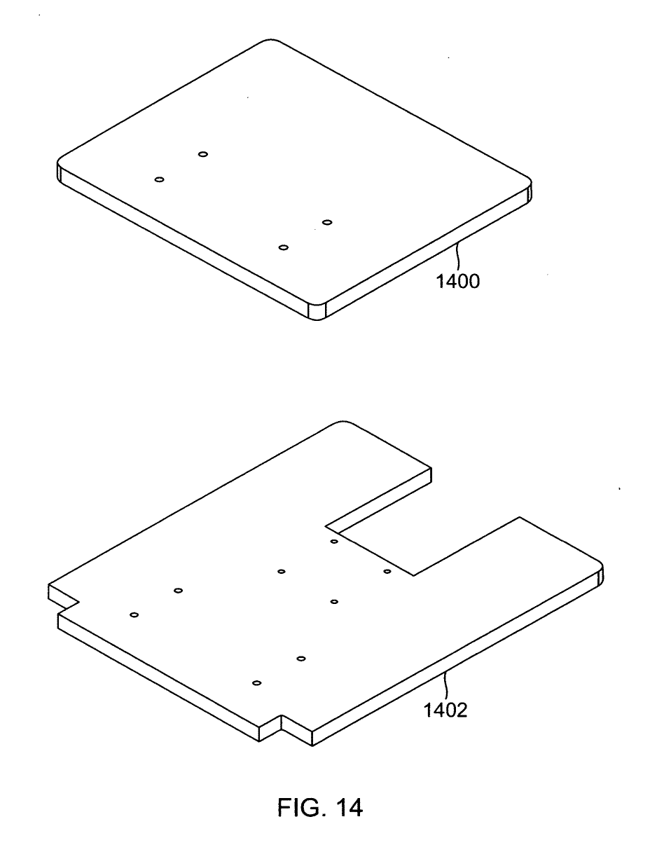

FIG. 10 shows different views of a seat assembly (1000) of a second traction system according to the present invention. FIG. 11 shows two different adjustable positions of a seat back platform (1100) connected to a seat back assembly of a second traction system according to the present invention. FIGS. 12-14 show various elements of the seat assembly of the second traction system. FIG. 12 shows a pivot assembly (1200, top image) and a seat back platform (1202, bottom image) of a seat assembly of a second traction system according to the present invention. FIG. 13 shows a sliding assembly (1300, top image) and a locking assembly (1302, bottom image) of a seat assembly of a second traction system according to the present invention. FIG. 14 shows a seat platform (1400, top image) and a leg platform (1402, bottom image) of a seat assembly of a second traction system according to the present invention.

The present invention is furthermore directed to corrective methods using spinal traction systems of the present invention. The systems are full spine corrective traction systems designed to correct and restore cervical, thoracic, lumbo-sacral and full spine spinal alignment that falls outside of peer reviewed, published medical normative values. The vertical and horizontal overhead system allows the spine to be pulled in every and any vector the human spine moves in three dimensional space. Traction in multiple planes is permitted by the system, and simultaneous traction can be applied to multiple regions of the spine in two or three dimensions. The seat assembly of the second traction system allows for a patient to be seated upright, supine and every angle and vector between 90 degrees upright and 0 degrees horizontal—e.g., between 90 degrees and 0 degrees, between 80 degrees and 10 degrees, between 70 degrees and 20 degrees, between 60 degrees and 30 degrees, and between 50 degrees and 40 degrees.

A patient who is a candidate for the traction system of the present invention typically reports pain in his back or neck. The patient is subjected to appropriate examinations, and x-rays are taken to determine whether the pain is caused by a spinal abnormality. Once a spinal abnormality is found, the patient is brought to the corrective traction system. While seated, standing or lying down, the patient is wrapped with a suitable harness (e.g., on head, around chest, around shoulders, etc.). The harness is connected to the pulley system of the traction device through a cable. Weights (e.g., 5 or 10 pounds) are added to the traction device such that a pulling force is applied to the area of interest. Within 10 minutes or less—e.g., 10 minutes or less, 9 minutes or less, 8 minutes or less, 7 minutes or less, 6 minutes or less, or 5 minutes or less—the force is removed, which allows ligaments to remain in the elastic phase. The described traction treatment is typically repeated once, twice, three times or four times per week over the period of a month. After approximately one month of treatment, a patient typically receives physical therapy for two more months involving exercises on the traction device. This therapy moves the subject ligament(s) from the elastic phase to the plastic phase, accordingly reshaping the patient's spine, a term known as biorheology.

The normal range for cervical spine lordosis is 34 to 42 degrees, while the normal range for lumbar lordosis is 36 to 40 degrees. Abnormal lordosis has been typically treated through surgery (e.g., vertebral fusion). Such surgery, however, mays not restore the spinal to normal lordosis ranges and it is expensive (e.g., $100,000). Use of the systems and methods described above typically restores the spinal to normal lordosis ranges for less than ¼^{th} the cost of surgery. Oftentimes the cost is less than ⅕^{th}, ⅙^{th}, 1/7^{th}, ⅛^{th}, 1/9^{th} or 1/10^{th} the cost of surgery. In certain cases, the spine lordosis is changed at least 2 degrees, 3 degrees, 4 degrees, or 5 degrees to reach the normal range. Also in certain cases, the lumbar lordosis is changed at least 2 degrees, 3 degrees, 4 degrees, or 5 degrees to reach the normal range.

Treatment of a patient with the corrective traction system of the present invention over a period of at least one month oftentimes decreases the patient's pain, as measured on the Visual Analog Scale (“VAS”) from 7-10 to 0 to 3. In certain cases, the VAS score is reduced from 7-10 to 3, 2, 1 or 0.

The corrective traction system of the present invention furthermore treats spinal conditions that have been associated with pulmonary and cardiovascular symptoms associated with abnormal lordosis and kyphosis ranges. Pulmonary symptoms include pulmonary hypertension and decreased lung capacity, while cardiovascular symptoms include the backing up of blood in the lungs and legs. For instance, treatment of a patient over a period of at least one month may increase lung capacity by at least 10 percent, 15 percent or 20 percent. A month's long treatment may reduce pulmonary hypertension (blood pressure of vessels in the lung) by at least 10 percent, 20 percent, 30 percent or 40 percent. Similar reductions in blood backup may also be seen—e.g., at least 10 percent, 20 percent, 30 percent, 40 percent or 50 percent reduction.

1. A spine corrective traction system, wherein the system comprises an overhead system allowing a patient's spine to be pulled in every and any vector the human spine moves in three dimensional space and a seat assembly that allows for a patient to be seated upright, supine and every angle and vector between 90 degrees upright and 0 degrees horizontal.

2. A method of correcting cervical or lumbar lordosis in a patient such that the normal range for lordosis is achieved, wherein the method comprises the steps of:
a) wrapping the patient in a harness, where the patient is standing, supine or seated at an angle less than 90 degrees;
b) pulling the harness in more than one vector at the same time for a period greater than 10 minutes, not to exceed 20 minutes;
c) releasing the harness;
d) repeating steps “a”, “b” and “c” at least three per week for 12 weeks thereby achieving a normal range of cervical or lumbar lordosis in the patient.

3) A method of treating thoracic hyperkyphosis which has been associated with pulmonary hypertension in a patient, wherein the method comprises:
a) wrapping the patient in a harness, where the patient is standing, supine or seated at an angle less than 90 degrees;
b) pulling the harness in more than one vector at the same time for a greater than 10 minutes, not to exceed 20 minutes;
c) releasing the harness
d) repeating steps “a”, “b” and “c” at least three times per week for 12 weeks thereby reducing the patient's thoracic hyperkyphosis which has been associated with pulmonary hypertension by at least 10 percent.

4) A method of increasing a patient's lung capacity, wherein the method comprises:
a) wrapping the patient in a harness, where the patient is standing, supine or seated at an angle less than 90 degrees;
b) pulling the harness in more than one vector at the same time for greater than 10 minutes, not to exceed 20 minutes;
c) releasing the harness;
d) repeating steps “a”, “b” and “c” at least three times per week for 12 weeks;
thereby increasing the patient's lung capacity by at least 10 percent.

5. A method of decreasing the cost of treating a patient having abnormal cervical spine lordosis or lumbar lordosis relative to surgical intervention, wherein the method comprises:
a) wrapping the patient in a harness, where the patient is standing, supine or seated at an angle less than 90 degrees;
b) pulling the harness in more than one vector at the same time for a greater than 10 minutes not to exceed 20 minutes;
c) releasing the harness;
d) repeating steps “a”, “b” and “c” at least once per week for one month thereby restoring cervical spine lordosis or lumbar lordosis to a normal range, and whereby the cost of performing the method is less than ¼^{th} the cost of surgical intervention.
